(19) United States
(12) Patent Application Publication
(10) Pub. No.: US 2012/0260934 A1
(43) Pub. Date: Oct. 18, 2012

(54) HAIR TREATMENT AGENTS COMPRISING TRIALKOXYSILANE-SUBSTITUTED COMPOUNDS AND ALKOXYSILYL-MODIFIED MACRO MOLECULES

(76) Inventors: Matthias Schweinsberg, Hamburg (DE); Winfried Emmerling, Tornesch (DE); Lars Zander, Rommerskirchen (DE)

(21) Appl. No.: 13/537,259

(22) Filed: Jun. 29, 2012

Related U.S. Application Data
(63) Continuation of application No. PCT/EP2010/069148, filed on Dec. 8, 2010.

(30) Foreign Application Priority Data
Dec. 30, 2009 (DE) 10 2009 055 404.1

(51) Int. Cl. A45D 7/04; A61K 8/86; A61Q 5/00; A61K 8/58

(52) U.S. Cl. 132/203; 424/70.1; 424/70.122

(57) ABSTRACT

Agents for treating keratin-containing fibers, particularly human hair, containing in a cosmetically acceptable carrier (i) at least one macromolecule having at least one alkoxysilyl functionality of the formula (RO)ₙR′₍₃₋ₙ₎Si—*, wherein R and R′ are independently a (C₁ to C₄) alkyl group (particularly methyl or ethyl), x is 1, 2 or 3, and (ii) at least one trialkoxysilane of the formula (T-1), wherein R¹ is a (C₁ to C₄) alkyl group, K′ is a connectivity chosen from a covalent bond or from a molecular fragment having two free valences, R² is a structural fragment having at least one substituent chosen from quaternary ammonium group, tertiary amino group, secondary amino group, primary amino group, anionic group, lead to a good washing resistance of the hairstyle fixed with the agent.

### CROSS REFERENCE TO RELATED APPLICATIONS

The present application is a continuation of International Application No. PCT/EP2010/069148 filed 8 Dec. 2010, which claims priority to German Patent Application No. 10 2009 055 404.1, filed 30 Dec. 2009, both of which are incorporated herein by reference.

### BACKGROUND OF THE INVENTION

The present invention relates to agents for hair treatment containing at least one trialkoxysilane-substituted compound and at least one alkoxysilyl-modified macromolecule, use of those agents for temporary shaping and/or care of keratin-containing fibers, and a corresponding method of application.

Today an attractive-looking hairstyle is regarded as an essential element of a well-groomed exterior. The latest fashion trends mean that for many hair types, hairstyles that are considered fashionable can be constructed or maintained for a lengthy period of up to only several days using active fixing agents. Therefore, hair treatment agents that provide a permanent or temporary shaping of the hair have an important role to play. Temporary shaping effects which offer good hold without adversely affecting the healthy appearance of the hair, such as its shine, can be achieved using hair sprays, hair waxes, hair gels, hair foams, blow-drying, etc.

Lasting shaping of the hair is performed, for example, by known permanent wave methods, by mechanically shaping the hair and fixing the shape by winding onto rollers or foam curlers, or by using a curling iron. Before, during or after this shaping the hair is treated with the aqueous preparation of a keratin-reducing substance, and, at the end of a contact period, rinsed with water or an aqueous solution. Sometimes it is advantageous to support the shaping using a hood dryer as a heat source. In a subsequent step, the hair is then treated with the aqueous preparation of an oxidizing agent. At the end of a contact period this too is rinsed out of the hair and the hair is freed from the mechanical shaping auxiliary agents (rollers, foam curlers).

Permanent straightening of keratin-containing fibers is achieved in an analogous manner with the use of keratin-reducing and -oxidizing compositions. In a corresponding method the curly hair is either wound onto curlers having a large diameter typically exceeding 15 mm, or the hair is combed straight under the influence of the keratin-reducing composition. Instead of rollers, it is also possible to lay the fiber flat on a straightening board. Straightening boards are typically rectangular boards made from, for example, plastic. The fiber is preferably wetted with the keratin-reducing preparation.

In addition to having a high degree of hold, styling agents also have to satisfy a whole series of additional requirements. These include properties of the hair, properties of the individual formulation (e.g., properties of the foam, gel or sprayed aerosol), and properties relating to the handling of the styling agent, with particular importance being attached to the properties of the hair. Mention can be made in particular of moisture resistance, low tackiness, and a balanced conditioning effect. As far as possible, a styling agent should also be universally suitable for all hair types.

In order to meet the diverse requirements, a large number of synthetic polymers for use in styling agents have been developed. These polymers can be divided into cationic, anionic, non-ionic and amphoteric film-forming and/or fixing polymers. Even when used in small amounts, the polymers ideally form a polymer film when applied to the hair, giving the hairstyle a strong hold while being sufficiently flexible so not to break under stress. If the polymer film is too brittle, film flakes or residues are formed which detach when the hair is moved, giving the impression that the user of the corresponding styling agent has dandruff.

### SUMMARY OF THE INVENTION

The present invention therefore provides an agent for shaping keratin-containing fibers having a high degree of hold and achieving a wash-resistant shaping result.

The present invention thus firstly provides an agent for treating keratin-containing fibers, particularly human hair, containing in a cosmetically acceptable carrier—
(i) at least one macromolecule having at least one alkoxysilyl functionality of the formula (RO)ₙR′₍₃₋ₙ₎Si—*, wherein R and R′ are independently a (C₁ to C₄) alkyl group (particularly methyl or ethyl), and n is 1, 2 or 3,
   and
(ii) at least one trialkoxysilane of the formula (T-1)

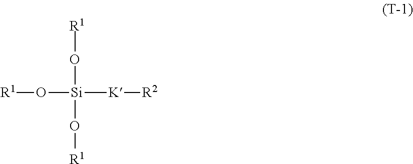

wherein
R¹ is a (C₁ to C₄) alkyl group,
K′ is a connectivity chosen from a covalent bond or from a molecular fragment having two free valences, and
R² is a structural fragment having at least one substituent chosen from quaternary ammonium group, tertiary amino group, secondary amino group, primary amino group, anionic group.

### DETAILED DESCRIPTION OF THE INVENTION

Keratin-containing fibers in principle include all animal hair, for example, wool, horsehair, angora hair, fur, feathers and products or textiles manufactured therefrom. The keratinic fibers are, however, preferably human hair.

In the above formula and all subsequent formulae, a chemical bond marked with the symbol * denotes a “free valence” of the corresponding structural fragment.

A “macromolecule” within the meaning of the invention is a molecule having a molar mass of at least 3000 g/mol. This includes inter alia polymers and dendrimers.

The alkoxysilyl-functionalized macromolecules are preferably present in agents according to the invention in an amount from 0.01 to 29.99 wt. %, more preferably from 0.1 to 14.9 wt. %, even more preferably 0.1 to 9.5 wt. %, and most preferably from 0.2 to 3.0 wt. %, based on total weight of the agent.

Preferred macromolecules are based on polymers such as polysaccharides (e.g., cellulose or starch) or polymers starting from ethylenically unsaturated monomers (e.g., acrylic acid, methacrylic acid, 2-hydroxyethyl acrylic acid, 2-hydroxyethyl methacrylic acid, acrylamide, meth acrylamide, 2-hydroxyethyl acrylamide, 2-hydroxyethyl methacrylamide), wherein these polymers are functionalized with at least one alkoxysilyl group of the formula (RO)ₙR′₍₃₋ₙ₎Si—*, wherein R and R′ are independently a (C₁ to C₄) alkyl group (particularly methyl or ethyl), and n is 1, 2 or 3. Preferred polymers have at least one structural unit of the formula (T-2),

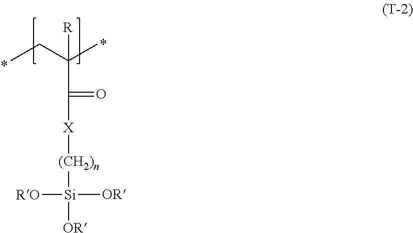

wherein
R is a hydrogen atom or a methyl group,
n is 2 or 3,
R′ is methyl or ethyl, and
X is an oxygen atom or an NH group.

Within the meaning of the invention, however, good effects have been achieved if the alkoxysilyl-functionalized macromolecule is chosen from alkoxysilyl-functionalized, polyether-modified compounds. Of these, alkoxysilyl-functionalized, polyether-modified organic compounds are preferably chosen that have at least three polyether substituents, the polyethers having a polyoxyalkylene chain comprising ethylene oxide units or ethylene oxide and propylene oxide units with a maximum proportion of 50 wt. % of propylene oxide units, based on weight of the polyoxyalkylene chain.

A “polyether substituent” is a chemical structural fragment having a polyoxyalkylene chain consisting of ethylene oxide units or ethylene oxide and propylene oxide units with a maximum proportion of 50 wt. % of propylene oxide units, based on weight of the polyoxyalkylene chain, which is covalently bonded to an organic compound either directly or via a chemical bond-forming structural fragment.

Within the meaning of the present invention an ethylene oxide unit is understood to be a unit of general formula (1)—

*—CH₂—CH₂—O—* Formula (1)

and within the meaning of the present invention a propylene oxide unit is understood to be a unit of general formula (2)

*—CH₂—CH(CH₃)—O—* Formula (2).

“Organic compounds” are chemical compounds based on a hydrocarbon structural fragment. Corresponding hydrocarbon structural fragments are derived from linear, branched, cyclic or heterocyclic hydrocarbons, each of which may be saturated, unsaturated or aromatic.

“Polyether-modified organic compounds” are according to the invention organic compounds that are modified with polyether-containing substituents, the polyether substituents in each case forming a chemical bond with the organic compound to be modified.

If the polyoxyalkylene chain of the polyether substituent contains ethylene oxide and propylene oxide units, the maximum proportion of propylene oxide units is preferably 40 wt. % and more preferably at most 30 wt. %, based on weight of A.

Alkoxysilyl-functionalized, polyether-modified, organic compounds having at least three polyether substituents are preferably chosen from at least one compound of the general formula (PE-1)—

[QK′-A-K-T)ₙ (PE-1)
wherein
A is a polyoxyalkylene chain comprising ethylene oxide units or ethylene oxide and propylene oxide units, having a maximum proportion of 50 wt. % of propylene oxide units, relative to the weight of A,
K and K′ are independently a connectivity chosen from a covalent bond or from a molecular fragment having two free valences,
T is a —Si(OR)ₓ(R′)₃₋ₓ residue wherein R and R′ are independently a (C₁ to C₄) alkyl group (particularly methyl or ethyl), and x is 1, 2 or 3,
Q is an organic structural fragment derived from linear, branched, cyclic or heterocyclic hydrocarbons, each of which may be saturated, unsaturated or aromatic, and
n is an integer number from 3 to 64 (particularly 3, 4, 5, 6, 7 or 8).

According to formula (PE-1), A is preferably a structural fragment of formula (A1)—

*—(OCH₂CH₂)ₚ—(OCH₂CH(CH₃))ₘ—* (A1)

wherein

p is an integer number from 1 to 500, and

m is an integer number from 0 to 500, and

the structural fragment of formula (A1) has a maximum proportion of 50 wt. % of propylene oxide units based on total weight of the structural fragment (A1). Ethylene oxide and propylene oxide units according to formula (PE-1) or according to formula (A1) can be randomly distributed or distributed as a gradient or can be present in at least two blocks.

If the group A of compounds according to formula (PE-1) (or according to all subsequent formulae containing A) is a polyoxyalkylene chain consisting of ethylene oxide and propylene oxide units, the maximum proportion of propylene oxide units is preferably 40 wt. %, and more preferably at most 30 wt. %, based on total weight of A.

K or K′ according to formula (PE-1) are preferably independently a covalent bond, an oxy group, a (C₁ to C₆) alkylene group, an imino group or at least one of the following connectivities (K1) to (K10)—

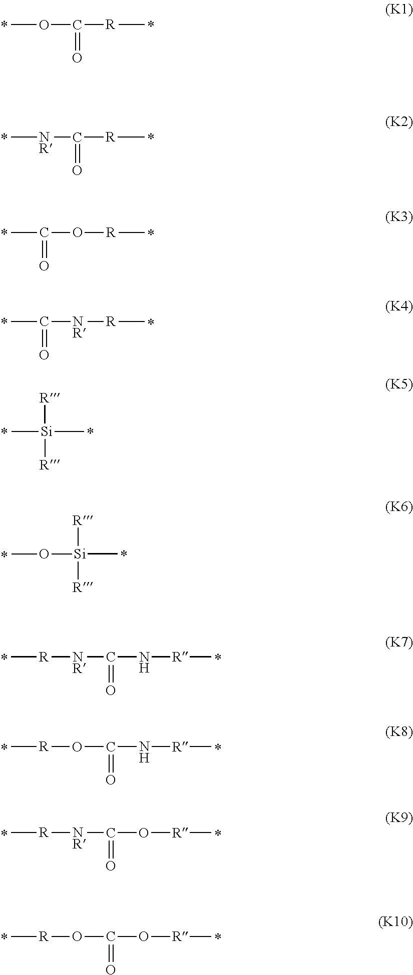

wherein
R and R″ are independently methylene, ethane-1,2-diyl, propane-1,2-diyl, propane-1,3-diyl, butane-1,2-diyl, butane-1,3-diyl, butane-1,4-diyl or phenylene,
R′ is a hydrogen atom or a (C₁ to C₄) alkyl group, and
R′″ independently of one another is a (C₁ to C₄) alkyl group or an aryl group.

T according to formula (PE-1) preferably is a —Si(OR)ₓ(R′)₃₋ₓ residue, wherein R and R′ are independently a (C₁ to C₄) alkyl group (particularly methyl or ethyl), and x is 2 or 3 (preferably triethoxysilyl).

The structural fragment —K-T according to formula (PE-1) or according to the following formulae (PE-1a) to (PE-1f) most preferably is the group—

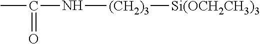

Q according to formula (PE-1) is preferably a corresponding organic residue having 2 to 30 carbon atoms, more preferably 2 to 20 carbon atoms.

Q is preferably derived from glycerol, monosaccharide, or disaccharide. Q is most particularly preferably derived from a compound chosen from sorbitol, 1,5-anhydrosorbitol, 1,4-anhydrosorbitol, inositol, xylitol, mannitol, gluconolactone, glucuronic acid, 1,2,6-hexanetriol, hydroxyethyl sorbitol, phytantriol, hydroxypropyl phytantriol, lactitol, maltitol, pentaerythritol, polyglycerol-3, glucose, fructose, galactose, ribose, xylose, mannose, sucrose, cellobiose, gentiobiose, isomaltose, lactose, lactulose, maltose, maltutose, melibiose, trehalose, nigerose, palatinose, rutinose, arabinose.

Most particularly preferred alkoxysilyl-functionalized, polyether-modified organic compounds are selected from at least one compound of formula (PE-1a) or (PE-1b) or (PE-1c) or (PE-1d) or (PE-1e) or (PE-1f) or mixtures thereof,

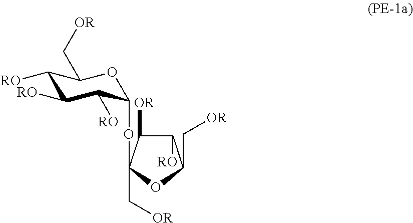

wherein
at least three R groups are a —(CH₂CH₂O)ₚ—(CHCH₃CH₂O)ₘ—K-T group and the other R groups are a hydrogen atom or a —K-T group, wherein independently of one another
p is an integer from 1 to 500, m an integer from 0 to 500, and p and m have a ratio to one another such that there is a maximum proportion of 50 wt. % (preferably a maximum of 40 wt. %, more preferably a maximum of 30 wt. %) of propylene oxide units, based on weight of the corresponding polyoxyalkylene chain,
K is a connectivity chosen from a covalent bond or from a molecular fragment having two free valences, and
T is a *—Si(OR)ₓ(R′)₃₋ₓ residue wherein R and R′ are independently a (C₁ to C₄) alkyl group (particularly methyl or ethyl), and x is 1, 2 or 3;

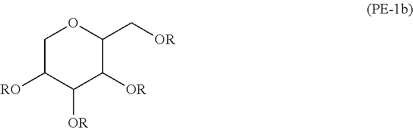

wherein
at least three R groups are a —(CH₂CH₂O)ₚ—(CHCH₃CH₂O)ₘ—K-T group and the other R groups are a hydrogen atom or a —K-T group, wherein independently of one another
p is an integer from 1 to 500, m an integer from 0 to 500, and p and m have a ratio to one another such that there is a maximum proportion of 50 wt. % (preferably a maximum of 40 wt. %, more preferably a maximum of 30 wt. %) of propylene oxide units, based on weight of the corresponding polyoxyalkylene chain,
K is a connectivity chosen from a covalent bond or from a molecular fragment having two free valences, and
T is a —Si(OR)ₓ(R′)₃₋ₓ residue wherein R and R′ are independently a (C₁ to C₄) alkyl group (particularly methyl or ethyl), and x is 1, 2 or 3;

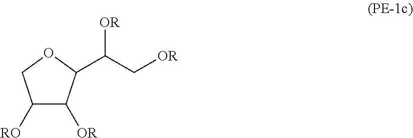

wherein
at least three R groups are a —(CH₂CH₂O)ₚ—(CHCH₃CH₂O)ₘ—K-T group and the other R groups are a hydrogen atom or a —K-T group, in which independently of one another
p is an integer from 1 to 500, m an integer from 0 to 500, and p and m have a ratio to one another such that there is a maximum proportion of 50 wt. % (preferably a maximum of 40 wt. %, more preferably a maximum of 30 wt. %) of propylene oxide units, based on weight of the corresponding polyoxyalkylene chain,
K is a connectivity chosen from a covalent bond or from a molecular fragment having two free valences, and
T is a —Si(OR)ₓ(R′)₃₋ₓ residue wherein R and R′ are independently a (C₁ to C₄) alkyl group (particularly methyl or ethyl), and x is 1, 2 or 3;

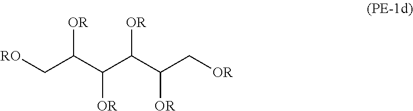

wherein
at least three R groups are a —(CH₂CH₂O)ₚ—(CHCH₃CH₂O)ₘ—K-T group and the other R groups are a hydrogen atom or a —K-T group, in which independently of one another
p is an integer from 1 to 500, m an integer from 0 to 500, and p and m have a ratio to one another such that there is a maximum proportion of 50 wt. % (preferably a maximum of 40 wt. %, more preferably a maximum of 30 wt. %) of propylene oxide units, based on weight of the corresponding polyoxyalkylene chain,
K is a connectivity chosen from a covalent bond or from a molecular fragment having two free valences, and
T is a —Si(OR)ₓ(R′)₃₋ₓ residue wherein R and R′ are independently a (C₁ to C₄) alkyl group (particularly methyl or ethyl), and x is 1, 2 or 3;

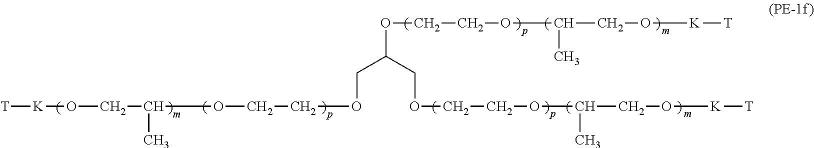

wherein independently of one another
p is an integer from 1 to 500, m an integer from 0 to 500 and p and m have a ratio to one another such that there is a maximum proportion of 50 wt. % (preferably a maximum of 40 wt. %, more preferably a maximum of 30 wt. %) of propylene oxide units, based on weight of the corresponding polyoxyalkylene chain,
K independently of one another is a connectivity chosen from a covalent bond or from a molecular fragment having two free valences, and
T is a —Si(OR)ₓ(R)₃₋ₓ residue wherein R and R′ are independently a (C₁ to C₄) alkyl group (particularly methyl or ethyl), and x is 1, 2 or 3.

Ethylene oxide and propylene oxide units can be randomly distributed or distributed as a gradient or can be present in at least two blocks.

The previously preferred groups K and T apply for formulae (PE-1a) to (PE-1f). The structural fragment *—K-T according to formula (PE-1a) to (PE-1f) is most preferably—

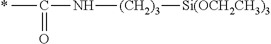

In addition to the alkoxysilyl-functionalized macromolecular compounds, agents according to the invention contain at least one trialkoxysilane of the above formula (T-1). These trialkoxysilanes differ from the macromolecules of feature (a). They therefore have a molecular weight of 3000 g/mol or less, particularly 1000 g/mol or less.

Trialkoxysilanes of the above formula (T-1) are preferably present in agents according to the invention in an amount from 0.01 to 29.99 wt. %, more preferably from 0.1 to 14.9 wt. %, even more preferably 0.1 to 9.5 wt. %, and most preferably from 0.2 to 3.0 wt. %, based on total weight of the agent.

Preferred trialkoxysilane compounds of the above formula (T-1) are those wherein R′ of formula (T-1) is methyl or ethyl, preferably ethyl.

Connectivity K′ according to formula (T-1) is preferably (C₂ to C₆) alkylene (particularly ethane-1,2-diyl or propane-1,3-diyl) or one of the formulae (K11), (K12) or (K13)—

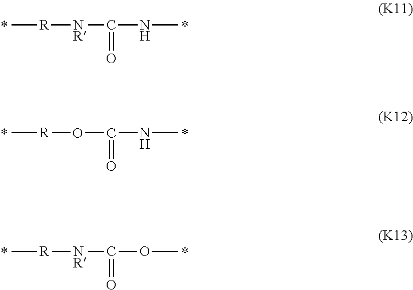

wherein
R is methylene, ethane-1,2-diyl, propane-1,2-diyl, propane-1,3-diyl, butane-1,2-diyl, butane-1,3-diyl, butane-1,4-diyl or phenylene (particularly ethane-1,2-diyl or propane-1,3-diyl), and
R′ is a hydrogen atom or a (C₁ to C₄) alkyl group.

Compounds of feature (ii) can be chosen from those bearing anionic groups in residue R². According to the invention, anionic groups are preferably a molecular fragment having 1 to 5, preferably 3, 4 or 5 deprotonatable acid groups. The anionic groups or deprotonatable acid groups of these molecular fragments are preferably chosen from carboxyl group and/or sulfonic acid group and/or phosphate or the salt forms thereof (particularly carboxyl group and/or sulfonic acid group or the salt forms thereof; particularly preferably carboxyl group or the salt form thereof).

In a particularly preferred embodiment, R² of formula (T-1) contains at least one, preferably at least two, more preferably 1 to 5, above all 2 to 5, particularly 2, 3, 4 or 5 carboxymethyl units. In a particularly preferred embodiment, the molecular fragment is an ethylene diamine triacetate unit that is covalently bonded to the connectivity K′ of formula (T-1) via one of its nitrogen atoms.

R² can further include a tertiary amino group, secondary amino group or primary amino group.

The groups 3-aminopropyl, 2-aminoethyl, aminomethyl, N,N-3-dimethylaminopropyl, N,N-2-dimethylaminoethyl, N,N-dimethylaminomethyl, N,N-3-diethylaminopropyl, N,N-2-diethylaminoethyl or N,N-diethylaminomethyl are preferably suitable structural fragments *—K′—R² of formula (T-1). The structural fragment *—K′—R² of formula (T-1) preferably is an H₂N—(CH₂)ₙ—* group with n=1, 2 or 3 (preferably with n=3).

In a most particularly preferred embodiment of the invention the trialkoxysilyl compounds of formula (T-1) are chosen from those having at least one quaternary ammonium residue in the residue R².

The groups N,N,N-3-trimethylammoniopropyl, N,N,N-2-trimethylammonioethyl, N,N,N-trimethylammoniomethyl, N,N,N-3-triethylammoniopropyl, N,N,N-2-triethylammonioethyl or N,N,N-triethylammoniomethyl are preferably suitable structural fragments *—K—R² of formula (T-1). The structural fragment *—K′—R² of formula (T-1) preferably is an Me₃N⁺-(CH₂)ₙ—* group with n=1, 2 or 3 (particularly with n=3).

Of those, it is in turn preferable for R² of formula (T-1) to be a group of formula (T-3),

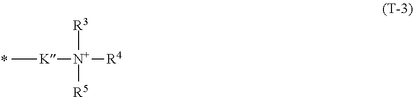

wherein
K″ is a direct bond or a (C₂ to C₆) alkylene group (particularly ethane-1,2-diyl),
R³ is methyl, ethyl or benzyl,
R⁴ and R⁵ are independently a methyl group, an ethyl group, a (C₈ to C₂₂) alkyl group, a (C₈ to C₂₂) alkenyl group, a 2-((C₈ to C₂₂) alkyl carbonyloxy)ethyl group, a 2-((C₈ to C₂₂) alkenyl carbonyloxy)ethyl group.

Most particularly preferred residues R² are derived from cationic surfactants. Of those, the structural fragments of formula (T-3) are in turn preferred wherein at least one of the groups R⁴ or R⁵ is a (C₈ to C₂₂) alkyl group, a (C₈ to C₂₂) alkenyl group, a 2-((C₈ to C₂₂) alkyl carbonyloxy)ethyl group or a 2-((C₈ to C₂₂) alkenyl carbonyloxy)ethyl group.

Trialkoxysilane compounds are preferably chosen from the following compounds of formula (T-1a)—

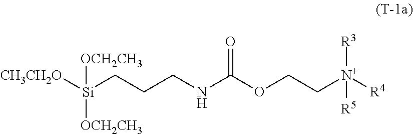

wherein
R³ is methyl, ethyl or benzyl,
R⁴ and R⁵ are independently a methyl group, an ethyl group, a (C₈ to C₂₂) alkyl group, a (C₈ to C₂₂) alkenyl group, a 2-((C₈ to C₂₂) alkyl carbonyloxy)ethyl group, a 2-((C₈ to C₂₂) alkenyl carbonyloxy)ethyl group, or a (C₈ to C₂₂) alkylamido (C₂ to C₃) alkyl group.

Of those, the trialkoxysilane compound is in turn preferably chosen from formulae (T-1b) to (T-1e)—

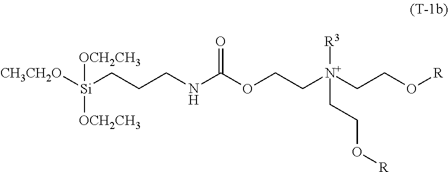

wherein R is a (C₈ to C₂₂) acyl group and R³ is methyl, ethyl or benzyl (particularly methyl);

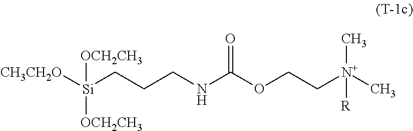

wherein R is a (C₈ to C₂₂) alkyl group;

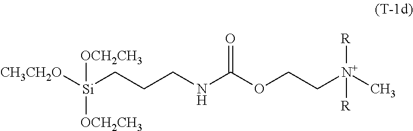

wherein each R is a (C₈ to C₂₂) alkyl group; and

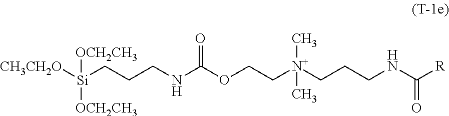

wherein R is a (C₈ to C₂₂) alkyl group.

Such compounds are obtained by reacting 3-triethoxysilyl propane isocyanate with a hydroxyl group-containing cationic surfactant (e.g., N-(2-hydroxyethyl)-N-methyl-N,N-bis(2-hexadecanoyloxyethyl)ammonium methosulfate (Dehyquart AU from Cognis)) in 1,4-dioxane as solvent or without the use of solvent.

Agents according to the invention additionally preferably contain as an activator at least one further compound chosen from organic amines having 2 to 20 carbon atoms, carboxylate complex compounds of tin, alkoxide compounds of tin, carboxylate complex compounds of lead, organoaluminum compounds, metal complexes of organic dicarbonyl compounds, and metal complexes of organic dicarboxylic acid esters. These activators are different from the alkoxysilanes of formula (T-1) and the alkoxysilane-modified macromolecular compounds.

Preferred activators are chosen from organic amines having 2 to 20 carbon atoms. Cyclic (particularly bicyclic) organic amines having at least 2 to 20 carbon atoms are in turn preferably suitable.

The organic amines are preferably chosen from at least one compound of the group comprising 1,4-diazabicyclo[2.2.2]octane (also referred to as DABCO), 1,8-diazabicyclo[5,4,0]undec-7-ene (also referred to as DBU), 1,5-diazabicyclo[4,3,0]non-5-ene (also referred to as DBN), n-butylamine, n-octylamine, lauryl amine, di-n-butylamine, 2-aminoethane sulfonic acid, N,N-2-dimethylaminoethane sulfonic acid, 2-aminoethanol, di-(2-hydroxyethyl)amine, tri-(2-hydroxyethyl)amine, diethylenetriamine, triethylenetetramine, oleylamine, cyclohexylamine, benzylamine, N,N-diethylaminopropylamine, xylylenediamine, triethylenediamine, guanidine, diphenylguanidine, 2,4,6-tris(dimethylaminomethyl)phenol, morpholine, N-methyl morpholine, and 2-ethyl-4-methylimidazole. It is in turn particularly preferable if additional activator is chosen from 1,4-diazabicyclo[2.2.2]octane (also referred to as DABCO), 1,5-diazabicyclo[4,3,0]non-5-ene (also referred to as DBN), and 1,8-diazabicyclo[5,4,0]undec-7-ene (also referred to as DBU).

Preferably suitable carboxylate complex compounds of tin are chosen from dibutyltin dilaurate (DBTL), dibutyltin diacetate, dibutyltin diethyl hexanoate, dibutyltin dioctoate, dibutyltin dimethyl maleate, dibutyltin diethyl maleate, dibutyltin dibutyl maleate, dibutyltin diisooctyl maleate, dibutyltin ditridecyl maleate, dibutyltin dibenzyl maleate, dibutyltin maleate, dibutyltin diacetate, tin octanoate, dioctyltin disteareate, dioctyltin dilaurate, dioctyltin diethyl maleate, dioctyltin diisooctyl maleate, dioctyltin diacetate, and tin naphthenoate.

Preferably, suitable alkoxide compounds of tin are chosen from dibutyltin dimethoxide, dibutyltin diphenoxide and dibutyltin diisoproxide.

Preferably suitable metal complexes of organic dicarbonyl compounds or metal complexes of organic dicarboxylic acid esters are chosen from aluminum trisacetylacetonate, aluminum trisethylacetoacetate, diisopropoxyaluminum ethyl acetoacetate, zirconium tetraacetylacetonate, and titanium tetraacetylacetonate.

Agents according to the invention contain activators preferably in an amount from 0.0001 to 10.0 wt. %, more preferably from 0.01 to 5.0 wt. %, based on total weight of the ready-to-use agent.

Agents according to the invention contain the ingredients and active ingredients in a cosmetically acceptable carrier.

Preferred cosmetically acceptable carriers are aqueous, alcoholic or aqueous-alcoholic media preferably having at least 10 wt. % water, based on total agent. Low alcohols having 1 to 4 carbon atoms that are typically used for cosmetic purposes, such as ethanol and isopropanol, can be included in particular as alcohols.

In a preferred embodiment, the agent therefore additionally contains at least one alcohol having 2 to 6 carbon atoms and 1 to 3 hydroxyl groups. This additional alcohol is preferably chosen from at least one compound of the group formed from ethanol, ethylene glycol, isopropanol, 1,2-propylene glycol, 1,3-propylene glycol, glycerol, n-butanol, 1,3-butylene glycol. A most particularly preferred alcohol is ethanol.

The additional alcohol having 2 to 6 carbon atoms and 1 to 3 hydroxyl groups is preferably present in the agent according to the invention in an amount from 20 wt. % to 65 wt. %, particularly 25 wt. % to 50 wt. %, based on total weight of the cosmetic agent.

Organic solvents or a mixture of solvents with a boiling point of 400° C. or less can be included as additional co-solvents in an amount from 0.1 to 15 wt. %, preferably 1 to 10 wt. %, based on total agent. Unbranched or branched hydrocarbons, such as pentane, hexane, isopentane, and cyclic hydrocarbons, such as cyclopentane and cyclohexane, are particularly suitable as additional co-solvents. A further particularly preferred water-soluble solvent is polyethylene glycol in an amount of up to 30 wt. % based on total agent.

In particular, the addition of polyethylene glycol and/or polypropylene glycol increases the flexibility of the polymer film formed on application of the agent according to the invention. If a flexible hold is desired, the agents therefore preferably contain 0.01 to 30 wt. % of polyethylene glycol and/or polypropylene glycol, based on total agent.

Agents according to the invention preferably additionally contain at least one surfactant, with non-ionic, anionic, cationic and ampholytic surfactants being suitable in principle. The group of ampholytic or amphoteric surfactants includes zwitterionic surfactants and ampholytes. According to the invention, the surfactants can already have an emulsifying action. Use of at least one non-ionic surfactant and/or at least one cationic surfactant is preferred within the context of this embodiment of the invention.

The additional surfactants are preferably present in the agent in an amount from 0.01 wt. % to 5 wt. %, more preferably from 0.05 wt % to 0.5 wt. %, based on total weight of the agent.

It is particularly preferable for agents according to the invention to additionally contain at least one non-ionic surfactant.

Non-ionic surfactants contain as a hydrophilic group, for example, a polyol group, a polyalkylene glycol ether group or a combination of a polyol and polyglycol ether group. Such compounds include—
addition products of 2 to 100 mol of ethylene oxide and/or 1 to 5 mol of propylene oxide with linear and branched fatty alcohols having 8 to 30 C atoms, with fatty acids having 8 to 30 C atoms and with alkyl phenols having 8 to 15 C atoms in the alkyl group;
addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as those available under the commercial names Dehydrol® LS, Dehydrol® LT (Cognis);
C₁₂-C₃₀ fatty acid monoesters and diesters of addition products of 1 to 30 mol of ethylene oxide with glycerol;
addition products of 5 to 60 mol of ethylene oxide with castor oil and hydrogenated castor oil;
polyol fatty acid esters, such as the commercial product Hydagen® HSP (Cognis) or Sovermol types (Cognis);
alkoxylated triglycerides;
alkoxylated fatty acid alkyl esters of formula (T-I)

R¹CO—(OCH₂CHR²)_{w}OR³ (T-I)
wherein R¹CO is a linear or branched, saturated and/or unsaturated acyl residue having 6 to 22 carbon atoms, R² is hydrogen or methyl, R³ is linear or branched alkyl residues having 1 to 4 carbon atoms, and w is a number from 1 to 20;
amine oxides;
hydroxy mixed ethers, such as are described in DE-OS 19738866;
sorbitan fatty acid esters and addition products of ethylene oxide with sorbitan fatty acid esters such as polysorbates;
sugar fatty acid esters and addition products of ethylene oxide with sugar fatty acid esters;
addition products of ethylene oxide with fatty acid alkanol amides and fatty amines;
sugar surfactants of the alkyl and alkenyl oligoglycoside type according to formula (T-II),

R⁴O-[G]ₚ (T-II)
wherein R⁴ is an alkyl or alkenyl residue having 4 to 22 carbon atoms, G is a sugar residue having 5 or 6 carbon atoms, and p is numbers from 1 to 10. They can be obtained by relevant methods of preparative organic chemistry. The alkyl and alkenyl oligoglycosides can derive from aldoses or ketoses having 5 or 6 carbon atoms, preferably from glucose. Preferred alkyl and/or alkenyl oligoglycosides are thus alkyl and/or alkenyl oligoglucosides. The index value p in the general formula (T-II) indicates the degree of oligomerization (DP) (i.e., the distribution of mono- and oligoglycosides), and is a number from 1 to 10. While p in the individual molecule must be an integer and can assume the values p=1 to 6 here, the value p for a particular alkyl oligoglycoside is a calculated quantity determined by analysis, which in most cases represents a fraction. Alkyl and/or alkenyl oligoglycosides having an average degree of oligomerization p of 1.1 to 3.0 are preferably used. From an application-oriented perspective, alkyl and/or alkenyl oligoglycosides having a degree of oligomerization of 1.7 or less, and particularly from 1.2 to 1.4 are preferred. The alkyl or alkenyl residue R⁴ can derive from primary alcohols having 4 to 11, preferably 8 to 10 carbon atoms. Typical examples are butanol, hexanol, octanol, decanol and undecanol as well as the technical mixtures thereof, such as are obtained in the hydrogenation of technical fatty acid methyl esters or during the hydrogenation of aldehydes from the Roden oxo synthesis. Preference is given to alkyl oligoglucosides with a chain length of C₈-C₁₀ (DP=1 to 3), which occur as the first flush in the separation by distillation of technical C₈-C₁₈ coconut fatty alcohol and which can be contaminated with a proportion of less than 6 wt. % of C₁₋₂ alcohol, as well as to alkyl oligoglucosides based on technical C_{9/11} oxo alcohols (DP=1 to 3). The alkyl or alkenyl residue R¹⁵ can also derive from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, as well as technical mixtures thereof, which can be obtained as described above. Alkyl oligoglucosides based on hydrogenated C_{12/14} coconut alcohol with a DP of 1 to 3 are preferred.

Alkylene oxide addition products with saturated linear fatty alcohols and fatty acids containing 2 to 100 mol of ethylene oxide per mol of fatty alcohol or fatty acid are most particularly preferred non-ionic surfactants. Preparations having outstanding properties are likewise obtained if they contain C₁₂-C₃₀ fatty acid mono- and diesters of addition products of 1 to 30 mol of ethylene oxide with glycerol and/or addition products of 5 to 60 mol of ethylene oxide with castor oil and hydrogenated castor oil as non-ionic surfactants.

Both products having a “normal” homolog distribution and those having a narrow homolog distribution can be used for the surfactants that are addition products of ethylene and/or propylene oxide with fatty alcohols or derivatives of these addition products. “Normal” homolog distribution refers to mixtures of homologs obtained by reacting fatty alcohol and alkylene oxide using alkali metals, alkali hydroxides or alkali alcoholates as catalysts. Narrow homolog distributions are obtained when, for example, hydrotalcites, alkaline-earth metal salts of ether carboxylic acids, alkaline-earth oxides, hydroxides or alcoholates are used as catalysts. Use of products having a narrow homolog distribution can be preferred.

Agents according to the invention most preferably contain at least one addition product of 15 to 100 mol of ethylene oxide, particularly 15 to 50 mol of ethylene oxide, with a linear or branched (particularly linear) fatty alcohol having 8 to 22 carbon atoms as the surfactant. This is preferably Ceteareth-15, Ceteareth-25 or Ceteareth-50, which are sold respectively as Eumulgin® CS 15 (COGNIS), Cremophor A25 (BASF SE) and Eumulgin® CS 50 (COGNIS).

All anionic surface-active substances suitable for use on the human body are suitable in principle as anionic surfactants. These have the characterizing feature of a water-solubilizing anionic group such as a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups.

Also suitable for use according to the invention are cationic surfactants of the quaternary ammonium compound, esterquat and amidoamine type. Preferred quaternary ammonium compounds are ammonium halides, particularly chlorides and bromides such as alkyl trimethylammonium chlorides, dialkyl dimethylammonium chlorides and trialkyl methylammonium chlorides. Long alkyl chains of these surfactants preferably have 10 to 18 carbon atoms, such as in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are the imidazolium compounds known under the INCI names Quaternium-27 and Quaternium-83.

Surface-active compounds classified as zwitterionic surfactants are those bearing at least one quaternary ammonium group and at least one —COO⁽⁻⁾ or —SO₃⁽⁻⁾ group in the molecule. Particularly suitable zwitterionic surfactants are betaines such as N-alkyl-N,N-dimethylammonium glycinates (e.g., cocoalkyl dimethylammonium glycinate), N-acyl aminopropyl-N,N-dimethylammonium glycinates (e.g., cocoacylaminopropyl dimethylammonium glycinate), and 2-alkyl-3-carboxymethyl-3-hydroxyethyl imidazolines, each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are surface-active compounds which, in addition to a C₈-C₂₄ alkyl or acyl group, contain at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytes are N-cocoalkyl aminopropionate, cocoacylaminoethyl aminopropionate and C₁₂-C₁₈ acyl sarcosine.

Agents according to the invention can also contain auxiliary substances and additives that are typically added to customary styling agents. Suitable auxiliary substances and additives include in particular additional care substances.

The agent can, for example, contain at least one protein hydrolysate and/or a derivative thereof as a care substance.

Protein hydrolysates are mixtures of products obtained by acidically, basically or enzymatically catalyzed breakdown of proteins. According to the invention, the term “protein hydrolysates” is also understood to include total hydrolysates and individual amino acids and derivatives thereof as well as mixtures of different amino acids. The term protein hydrolysates is further understood to include polymers synthesized from amino acids and amino acid derivatives. The latter include polyalanine, polyasparagine, polyserine, etc. Further examples of compounds that can be used according to the invention are L-alanyl-L-proline, polyglycine, glycyl-L-glutamine or D/L-methionine-5-methyl sulfonium chloride. β-Amino acids and derivatives thereof, such as β-alanine, anthranilic acid or hippuric acid, can also be used according to the invention. The molecular weight of protein hydrolysates for use according to the invention is from 75, the molecular weight for glycine, to 200,000. The molecular weight is preferably 75 to 50,000, and more preferably 75 to 20,000 Daltons.

According to the invention, protein hydrolysates of plant, animal, marine or synthetic origin can be used.

Animal protein hydrolysates include elastin, collagen, keratin, silk and milk protein hydrolysates, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

Protein hydrolysates of plant origin (e.g., soy, almond, pea, potato and wheat protein hydrolysates) are available, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although use of protein hydrolysates as such is preferred, amino acid mixtures obtained by other means can optionally also be used in their place. Use of derivatives of protein hydrolysates, for example, in the form of their fatty acid condensation products, is likewise possible. Such products are sold, for example, under the names Lamepon® (Cognis), Lexein® (Inolex), Crolastin® (Croda), Crosilk® (Croda) or Crotein® (Croda).

The teaching according to the invention naturally includes all isomeric forms, such as cis-trans isomers, diastereomers and chiral isomers. It is also possible according to the invention to use a mixture of several protein hydrolysates.

Protein hydrolysates can be present in agents according to the invention in concentrations, for example, from 0.01 wt. % to 20 wt. %, preferably 0.05 wt. % to 15 wt. % and more preferably 0.05 wt. % to 5 wt. %, based on total application preparation.

Agents according to the invention can further contain at least one vitamin, provitamin, vitamin precursor and/or derivative thereof as a care substance.

Such vitamins, provitamins and vitamin precursors that are conventionally assigned to groups A, B, C, E, F and H are preferred according to the invention.

The group of substances classified as vitamin A includes retinol (vitamin A₁) and 3,4-didehydroretinol (vitamin A₂). β-Carotene is the retinol provitamin. Suitable vitamin A components according to the invention include vitamin A acid and esters thereof, vitamin A aldehyde and vitamin A alcohol and esters thereof such as palmitate and acetate. Agents contain the vitamin A component preferably in amounts from 0.05 to 1 wt. %, based on total application preparation.

The vitamin B group or vitamin B complex includes inter alia—
Vitamin B₁ (thiamine);
Vitamin B₂ (riboflavin);
Vitamin B₃. The compounds nicotinic acid and nicotinic acid amide (niacinamide) are often included under this term. Preferred according to the invention is nicotinic acid amide, which is preferably present in agents according to the invention in amounts from 0.05 to 1 wt. %, based on total application preparation.
Vitamin B₅ (pantothenic acid, panthenol and pantolactone). Within the context of this group panthenol and/or pantolactone is preferably used. Derivatives of panthenol which can be used according to the invention include esters and ethers of panthenol as well as cationically derivatized panthenols. Individual representatives include panthenol triacetate, panthenol monoethyl ether and the monoacetate thereof, and cationic panthenol derivatives. The cited compounds of the vitamin B₅ type are preferably present in agents according to the invention in amounts from 0.05 to 10 wt. %, based on total application preparation. Amounts from 0.1 to 5 wt. % are particularly preferred.
Vitamin B₆ (pyridoxine as well as pyridoxamine and pyridoxal). The cited compounds of the vitamin B₆ type are preferably present in agents according to the invention in amounts from 0.01 to 5 wt. %, based on total application preparation. Amounts from 0.05 to 1 wt. % are particularly preferred.
Vitamin C (ascorbic acid). Vitamin C is used in the agents according to the invention preferably in amounts from 0.1 to 3 wt. %, based on total application preparation. Use in the form of palmitic acid ester, glucosides or phosphates can be preferred. Use in combination with tocopherols can likewise be preferred.
Vitamin E (tocopherols, particularly α-tocopherol). Tocopherol and derivatives thereof, which include in particular esters such as acetate, nicotinate, phosphate and succinate, are preferably present in agents according to the invention in amounts from 0.05 to 1 wt. %, based on total application preparation.
Vitamin F. The term “vitamin F” is typically understood to mean essential fatty acids, particularly linoleic acid, linolenic acid and arachidonic acid.
Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably present in agents according to the invention in amounts from 0.0001 to 1.0 wt. %, particularly 0.001 to 0.01 wt. %, based on total application preparation.

Agents according to the invention preferably contain vitamins, provitamins and vitamin precursors from groups A, B, C, E and H. Panthenol, pantolactone, pyridoxine and derivatives thereof as well as nicotinic acid amide and biotin are particularly preferred.

Like the addition of glycerol and/or propylene glycol, the addition of panthenol increases the flexibility of the polymer film formed from the application of the agent according to the invention. If a particularly flexible hold is desired, agents according to the invention can therefore contain panthenol instead of or in addition to glycerol and/or propylene glycol. In a preferred embodiment, agents according to the invention contain panthenol, preferably in an amount from 0.05 to 10 wt. %, more preferably 0.1 to 5 wt. %, based on total agent.

Agents according to the invention can also contain at least one plant extract as a care substance.

These extracts are typically produced by extraction of the entire plant. However, it can also be preferred in individual cases to produce the extracts exclusively from flowers and/or leaves of the plant.

Regarding preferred plant extracts according to the invention, reference is made in particular to the extracts listed in the table beginning on page 44 of the 3^{rd} edition of Leitfaden zur Inhaltsstoffdeklaration kosmetischer Mittel, published by the Industrieverband Körperpflege-und Waschmittel e.V. (IKW), Frankfurt.

Extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred according to the invention.

Extracts from green tea, oak bark, stinging nettle, witch hazel, hops, chamomile, burdock, horsetail, lime blossom, almond, aloe vera, coconut, mango, apricot, lemon, wheat, kiwi, melon, orange, grapefruit, sage, rosemary, birch, lady's smock, wild thyme, yarrow, restharrow, meristem, ginseng and ginger root are particularly preferred.

Extracts from green tea, almond, aloe vera, coconut, mango, apricot, lemon, wheat, kiwi and melon are even more particularly suitable.

Plant extracts can be used according to the invention in both pure and diluted form. If they are used in diluted form they typically contain approximately 2 to 80 wt. % of active substance and as the solvent, the extracting agent, or mixture of extracting agents used to obtain them.

It can also be preferred to use mixtures of a plurality of different plant extracts, particularly two, in agents according to the invention.

Agents according to the invention preferably contain these care substances in amounts from 0.001 to 2, particularly 0.01 to 0.5 wt. %, based on total application preparation.

With addition of a UV filter, both the agents and the treated fibers can be protected from damaging influences of UV radiation. At least one UV filter is therefore preferably added to the agent. There are no general restrictions on suitable UV filters in terms of their structure and physical properties. In fact, all UV filters that can be used in the cosmetics sector whose absorption maximum is in the UVA (315-400 nm), UVB (280-315 nm) or UVC (<280 nm) range are suitable. UV filters having an absorption maximum in the UVB range, particularly in the range from approximately 280 to approximately 300 nm, are particularly preferred.

Preferred UV filters include substituted benzophenones, p-aminobenzoic acid esters, diphenyl acrylic acid esters, cinnamic acid esters, salicylic acid esters, benzimidazoles and o-aminobenzoic acid esters.

Examples of UV filters that can be used according to the invention are 4-aminobenzoic acid, N,N,N-trimethyl-4-(2-oxoborn-3-ylidene methyl)aniline methyl sulfate, 3,3,5-trimethyl cyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and potassium, sodium and triethanolamine salts thereof (Phenylbenzimidazole sulfonic acid; Parsol®HS; Neo Heliopan®Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (Butyl methoxydibenzoylmethane; Parsol®1789, Eusolex®9020), α-(2-oxoborn-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (Octyl Dimethyl PABA; Uvasorb®DMO, Escalol®507, Eusolex®6007), salicylic acid-2-ethylhexyl ester (Octyl Salicylate; Escalol®587, Neo Heliopan®OS, Uvinul®O18), 4-methoxycinnamic acid isopentyl ester (Isoamyl p-Methoxycinnamate; Neo Heliopan®E 1000), 4-methoxycinnamic acid-2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene camphor; Parsol®5000, Eusolex®6300), 3-benzylidene camphor (3-Benzylidene camphor), 4-isopropylbenzyl salicylate, 2,4,6-trianilino-(p-carbo-2′-ethylhexyl-1′-oxi)-1,3,5-triazine, 3-imidazol-4-ylacrylic acid and ethyl esters thereof, polymers of N-{(2 and 4)-[2-oxoborn-3-ylidene methyl]benzyl}acrylamide, 2,4-dihydroxybenzophenone (Benzophenone-1; Uvasorb®20 H, Uvinul®400), 1,1′-diphenylacrylonitrilic acid-2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid-2′-ethylhexyl ester. Use of 4-methoxycinnamic acid isopentyl ester (Isoamyl p-Methoxycinnamate; Neo Heliopan®E 1000) is particularly preferred.

UV filters whose molar extinction coefficient at the absorption maximum is above 15,000, particularly above 20,000, are preferred.

UV filters are typically present in amounts from 0.01 to 5 wt. %, based on total application preparation. Amounts from 0.1 to 2.5 wt. % are preferred.

Further active ingredients, auxiliary substances and additives include—
thickening agents such as agar-agar, guar gum, alginates, xanthan gum, gum arabic, karaya gum, carob seed meal, linseed gums, dextrans, cellulose derivatives (e.g., methyl cellulose, hydroxyalkyl cellulose and carboxymethyl cellulose), starch fractions and derivatives such as amylose, amylopectin and dextrins, clays such as bentonite, fully synthetic hydrocolloids such as polyvinyl alcohol, and optionally crosslinked polyacrylates;
texturizing agents such as maleic acid and lactic acid;
perfume oils, dimethyl isosorbide and cyclodextrins;
defoaming agents such as silicones;
dyes to color the agent;
anti-dandruff active ingredients such as piroctone olamine, zinc omadine and climbazole;
substances to adjust the pH, such as conventional acids, particularly edible acids and bases;
consistency modifiers such as sugar esters, polyol esters or polyol alkyl ethers;
complexing agents such as EDTA, NTA, β-alanine diacetic acid and phosphonic acids;
swelling and penetrating substances such as glycerol, propylene glycol monoethyl ether, carbonates, hydrogen carbonates, guanidines, and ureas, as well as primary, secondary and tertiary phosphates;
opacifiers such as latex, styrene/PVP and styrene/acrylamide copolymers;
pearlescent agents such as ethylene glycol mono- and distearate as well as PEG-3 distearate;
preservatives; and
antioxidants.

Regarding further optional components and the amounts of these components used, reference is expressly made to the relevant manuals known to one skilled in the art.

For application on the keratin-containing fibers, the agent according to the invention is mixed from a first composition containing in a cosmetic carrier—
at least one macromolecule having at least one alkoxysilyl functionality of the formula (RO)ₙR′₍₃₋ₙ₎Si—*, wherein R and R′ are independently a (C₁ to C₄) alkyl group (particularly methyl or ethyl), and x is 1, 2 or 3,
   and a second composition containing in a cosmetic carrier—

at least one trialkoxysilane of the formula (T-1)

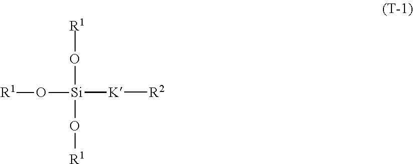

wherein
R¹ is a (C₁ to C₄) alkyl group,
K′ is a connectivity chosen from a covalent bond or from a molecular fragment having two free valences, and
R² is a structural fragment having at least one substituent chosen from quaternary ammonium group, tertiary amino group, secondary amino group, primary amino group, and an anionic group.
   With the presence of the two components, the resulting mixture gives rise to a reactive system that is responsible for the technical effect according to the invention.

Embodiments of the macromolecular compounds and compounds of formula (T-1) that were previously mentioned as being preferred are preferred here with necessary alterations.

The first composition preferably contains an alcoholic cosmetic carrier as the cosmetic carrier. Aqueous-alcoholic carriers are possible but less preferred. If water is present in the first composition, the amount thereof must be kept as low as possible. The first composition preferably contains 1.5 wt. % or less of free water. Low alcohols having 1 to 4 carbon atoms that are typically used for cosmetic purposes, such as ethanol and isopropanol, can be used in particular as alcohols.

In a preferred embodiment, the first composition therefore additionally contains at least one alcohol having 2 to 6 carbon atoms and 1 to 3 hydroxyl groups. This additional alcohol is preferably chosen from at least one compound of ethanol, ethylene glycol, isopropanol, 1,2-propylene glycol, 1,3-propylene glycol, glycerol, n-butanol, 1,3-butylene glycol. A most particularly preferred alcohol is ethanol.

The additional alcohol having 2 to 6 carbon atoms and 1 to 3 hydroxyl groups is preferably present in agents according to the invention in an amount from 20 wt. % to 65 wt. %, particularly from 25 wt. % to 50 wt. %, based on total first composition.

Organic solvents or a mixture of solvents with a boiling point of 400° C. or less can be included as additional co-solvents in an amount from 0.1 to 15 wt. %, preferably 1 to 10 wt. %, based on total agent. Unbranched or branched hydrocarbons such as pentane, hexane, and isopentane and cyclic hydrocarbons such as cyclopentane and cyclohexane, are particularly suitable as additional co-solvents. A further particularly preferred water-soluble solvent is polyethylene glycol in an amount of up to 30 wt. % based on total first composition.

A second subject-matter according to the invention is a kit of parts comprising—
in a first container, a composition containing in a cosmetic carrier at least one macromolecule having at least one alkoxysilyl functionality of the formula (RO)ₙR′₍₃₋ₙ₎Si—*, wherein R and R′ are independently a (C₁ to C₄) alkyl group (particularly methyl or ethyl), and x is 1, 2 or 3, and
in a second container, a composition containing in a cosmetic carrier at least one trialkoxysilane of the formula (T-1)

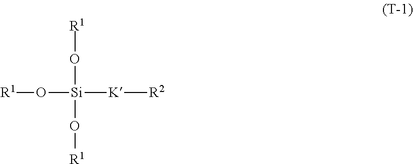

wherein
R¹ is a (C₁ to C₄) alkyl group,
K′ is a connectivity chosen from a covalent bond or from a molecular fragment having two free valences, and
R² is a structural fragment having at least one substituent chosen from quaternary ammonium group, tertiary amino group, secondary amino group, primary amino group, and anionic group.

The embodiments of the macromolecules or trialkoxysilanes of formula (T-1) that were previously mentioned as being preferred are preferred here with necessary alterations.

The two compositions are physically packaged separate from one another in separate containers. The individual containers can be, for example, bottles or cans made from materials such as plastic, metal or glass. A container within the meaning of the invention can also be a chamber of a multi-chamber receptacle. This multi-chamber receptacle can include a device that allows both compositions to be removed simultaneously from the multi-chamber receptacle. The compositions can be mixed together automatically before or after leaving the multi-chamber receptacle.

A third subject-matter of the invention is a method for lasting shaping of keratin-containing fibers, particularly human hair, wherein—
(i) the fibers are shaped with the aid of shaping auxiliary agents after, before or during step (ii),
(ii) an agent of the first subject-matter of the invention is brought into contact with the fibers, and
(iii) after a contact period, the fibers are optionally rinsed and dried.

Shaping auxiliary agents within the meaning of the method according to the invention include—
rolling aids such as curlers or foam rollers in the case of a permanent wave, or
auxiliary agents for mechanical straightening, such as a comb or a brush, a straightening board or a heatable straightening iron in the case of hair straightening.

If the shaping auxiliary agents (e.g., rolling aids) are attached to the fiber for an extended period of time in a waving process, it is convenient to remove these shaping auxiliary agents after step (iii).

The contact period is preferably 5 to 20 minutes, more preferably 10 to 15 minutes.

In a preferred embodiment, the keratin-containing fibers are moistened before step (i).

This can be done by spraying the fibers with a liquid (e.g., with water). Before step (i), the fibers are preferably shampooed with a conventional shampoo, rinsed, and then rubbed dry with a towel. After rubbing dry, a tangible residual moisture remains in the hair. For moistening purposes, it is also within the scope of the invention for an agent from the first or second container (see kit of parts in accordance with the second subject-matter of the invention) to be brought into contact with the hair. After the use of shaping auxiliary agents (particularly rolling aids), the agent from the kit of parts according to the invention that was not previously used for moistening is applied to the fibers, without rinsing the hair first. In this way, the agent according to the invention of the first subject-matter of the invention is formed on the keratin-containing fibers. The procedure is then continued in accordance with steps (ii) and (iii).

Preferred suitable embodiments of the agent according to the invention from step (ii) are mentioned in the first subject-matter of the invention (see above).

In a further embodiment of the method according to the invention, the hair is subjected to a heat treatment, particularly at temperatures from 80° C. to 250° C., more preferably from 120° C. to 200° C., before step (ii). A heated solid, preferably heated to 80° C. to 250° C., more preferably to 120° C. to 200° C., is preferably brought into direct contact with the hair as a heat source. This is the case when curling irons or straightening irons are used.

In a further variant of this embodiment as part of a hair straightening process, the fibers are subjected in step (iii) to a heat treatment with mechanical straightening of the fiber at a temperature of 120 to 220° C.

If curling irons are used, a strand of hair is wound around a correspondingly heated rounded body (e.g., a rod-shaped or tubular body) and, after a holding time (particularly 10 to 30 seconds), is unwound again.

According to the invention, a mechanical straightening is a stretching of curly hair along the longest spatial extension of the hair fiber.

Heat treatment with mechanical straightening of the hair preferably takes place at a temperature of 140 to 200° C. The heat treatment can take place with hot air. In this case, the hair is heated during combing at precisely the location wherein mechanical straightening occurs.

Depending on the manner of straightening, it is furthermore particularly preferable for the heat treatment to take place with the aid of heated plates, particularly metal or ceramic plates, wherein the plate is pressed against the hair to be straightened and then the pressed plate is moved along the hair fiber. The plates can be optionally coated with heat-resistant materials. The hair fiber straightened is particularly preferably pressed between two correspondingly heated plates and the two plates are moved together along the longest spatial extension of the fiber. It is preferred for both plates to be joined together so that the two plates can move evenly along the hair fiber. If the heat treatment is performed on living hair, the hair fiber is fixed at one end (hair root). In this case, the plates preferably move evenly along the entire hair fiber away from the hair root. This movement leads to a mechanical straightening of the fiber. A corresponding device for heat treatment is, for example, the Ceramic Flat-Master device (sold by Efalock, Germany).

We claim:

1. An agent for treating keratin-containing fibers comprising in a cosmetically acceptable carrier:
(i) at least one macromolecule bearing at least one alkoxysilyl functionality of the formula (RO)ₙR′₍₃₋ₙ₎Si—*, wherein R and R′ are independently a (C₁ to C₄) alkyl group, and n is 1, 2 or 3,
and
(ii) at least one trialkoxysilane of the formula (T-1) 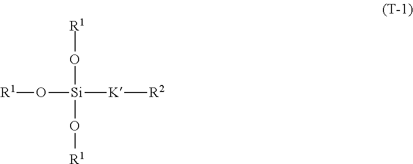
wherein
R′ is a (C₁ to C₄) alkyl group,
K′ is a connectivity chosen from a covalent bond or from a molecular fragment having two free valences, and
R² is a structural fragment having at least one substituent chosen from quaternary ammonium group, tertiary amino group, secondary amino group, primary amino group, anionic group.

2. The agent according to claim 1, wherein the at least one macromolecule is present in an amount from 0.01 to 29.99 wt. %, based on total weight of the agent.

3. The agent according to claim 1, wherein the at least one macromolecule is chosen from alkoxysilyl-functionalized polyether-modified compounds having at least three polyether substituents, polyethers having a polyoxyalkylene chain comprising ethylene oxide units or ethylene oxide and propylene oxide units with a maximum proportion of 50 wt. % of propylene oxide units, based on weight of the polyoxyalkylene chain.

4. The agent according to claim 1, wherein the at least one macromolecule is chosen from at least one compound of the general formula (PE-1)
[QK′-A-K-T)ₙ (PE-1)
wherein
A is a polyoxyalkylene chain having ethylene oxide units or ethylene oxide and propylene oxide units with a maximum proportion of 50 wt. % of propylene oxide units, relative to the weight of A,
K and K′ are independently a connectivity chosen from a covalent bond or from a molecular fragment having two free valences,
T is a —Si(OR)ₓ(R′)₃₋ₓ residue wherein R and R′ are independently a (C₁ to C₄) alkyl group (particularly methyl or ethyl), and x is 1, 2 or 3,
Q is an organic structural fragment derived from linear, branched, cyclic or heterocyclic hydrocarbons, all of which may each be saturated, unsaturated or aromatic, and
n is an integer number from 3 to 64.

5. The agent according to claim 1, wherein the at least one macromolecule is chosen from at least one compound of the formulae (PE-1a), (PE-1b), (PE-1c), (PE-1d), (PE-1e), (PE-1f) or mixtures thereof, 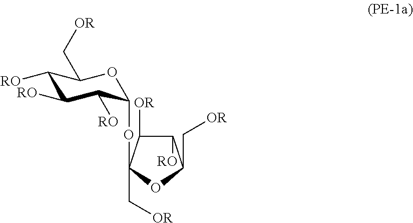
wherein
at least three R groups are a —(CH₂CH₂O)ₚ—(CHCH₃CH₂O)ₘ—K-T group and the other R groups are a hydrogen atom or a —K-T group, wherein independently of one another
p is an integer number from 1 to 500, m an integer number from 0 to 500, and p and m have a ratio to one another such that there is a maximum proportion of 50 wt. % of propylene oxide units, based on weight of the corresponding polyoxyalkylene chain,
K is a connectivity chosen from a covalent bond or from a molecular fragment having two free valences, and
T is a *—Si(OR)ₓ(R′)₃₋ₓ residue wherein R and R′ are independently a (C₁ to C₄) alkyl group, and x is 1, 2 or 3; 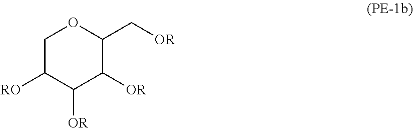
wherein
at least three R groups are a —(CH₂CH₂O)ₚ—(CHCH₃CH₂O)ₘ—K-T group and the other R groups are a hydrogen atom or a —K-T group, in which independently of one another
p is an integer number from 1 to 500, m an integer number from 0 to 500, and p and m have a ratio to one another such that there is a maximum proportion of 50 wt. % of propylene oxide units, based on weight of the corresponding polyoxyalkylene chain,
K is a connectivity chosen from a covalent bond or from a molecular fragment having two free valences, and
T is a —Si(OR)ₓ(R′)₃₋ₓ residue wherein R and R′ are independently a (C₁ to C₄) alkyl group, and x is 1, 2 or 3; 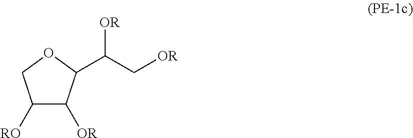
wherein
at least three R groups are a —(CH₂CH₂O)ₚ—(CHCH₃CH₂O)ₘ—K-T group and the other R groups are a hydrogen atom or a —K-T group, in which independently of one another
p is an integer number from 1 to 500, m an integer number from 0 to 500, and p and m have a ratio to one another such that there is a maximum proportion of 50 wt. % of propylene oxide units, based on weight of the corresponding polyoxyalkylene chain,
K is a connectivity selected from a covalent bond or from a molecular fragment having two free valences, and
T is a —Si(OR)ₓ(R′)₃₋ₓ residue wherein R and R′ are independently a (C₁ to C₄) alkyl group, and x is 1, 2 or 3; 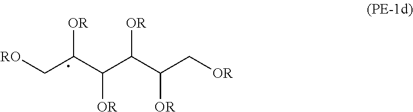
wherein
at least three R groups are a —(CH₂CH₂O)ₚ—(CHCH₃CH₂O)ₘ—K-T group and the other R groups are a hydrogen atom or a —K-T group, in which independently of one another
p is an integer number from 1 to 500 and m an integer number from 0 to 500 and p and m have a ratio to one another such that there is a maximum proportion of 50 wt. % (preferably a maximum of 40 wt. %, particularly preferably a maximum of 30 wt. %) of propylene oxide units, relative to the weight of the corresponding polyoxyalkylene chain,
K is a connectivity selected from a covalent bond or from a molecular fragment having two free valences, and
T is a —Si(OR)ₓ(R′)₃₋ₓ residue wherein R and R′ are independently a (C₁ to C₄) alkyl group, and x is 1, 2 or 3; 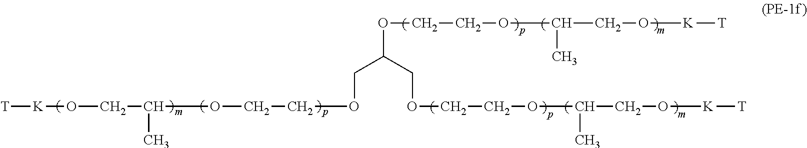
wherein independently of one another
p is an integer number from 1 to 500, m an integer number from 0 to 500, and p and m have a ratio to one another such that there is a maximum proportion of 50 wt. % of propylene oxide units, based on weight of the corresponding polyoxyalkylene chain,
K is independently a connectivity chosen from a covalent bond or from a molecular fragment having two free valences, and
T is a —Si(OR)ₓ(R′)₃₋ₓ residue wherein R and R′ are independently a (C₁ to C₄) alkyl group, and x is 1, 2 or 3.

6. The agent according to claim 1, wherein the at least one trialkoxysilane of formula (T-1) is present in an amount from 0.01 to 29.99 wt. %, based on total weight of the agent.

7. The agent according to claim 1, wherein K′ of formula (T-1) is (C₂ to C₆) alkylene or one of the formulae (K11), (K12) or (K13) 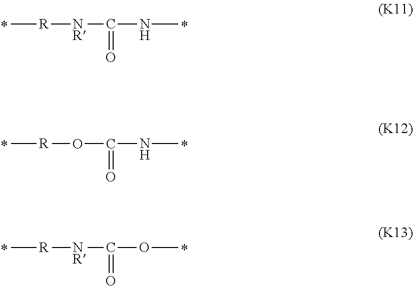
wherein
R is methylene, ethane-1,2-diyl, propane-1,2-diyl, propane-1,3-diyl, butane-1,2-diyl, butane-1,3-diyl, butane-1,4-diyl or phenylene, and
R′ is a hydrogen atom or a (C₁ to C₄) alkyl group.

8. The agent according to claim 1, wherein R² of formula (T-1) is a group according to formula (T-3), 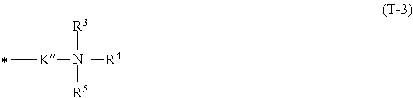
wherein
K″ is a direct bond or a (C₂ to C₆) alkylene group,
R³ is methyl, ethyl or benzyl,
R⁴ and R⁵ are independently a methyl group, an ethyl group, a (C₈ to C₂₂) alkyl group, a (C₈ to C₂₂) alkenyl group, a 2-((C₈ to C₂₂) alkyl carbonyloxy)ethyl group, or a 2-((C₈ to C₂₂) alkenyl carbonyloxy)ethyl group.

9. A kit of parts comprising:
in a first container, a composition comprising in a cosmetic carrier at least one macromolecule having at least one alkoxysilyl functionality of the formula (RO)ₙR′₍₃₋ₙ₎Si—*, wherein R and R′ are independently a (C₁ to C₄) alkyl group, and x is 1, 2 or 3,
and
in a second container, a composition comprising in a cosmetic carrier at least one trialkoxysilane of the formula (T-1) 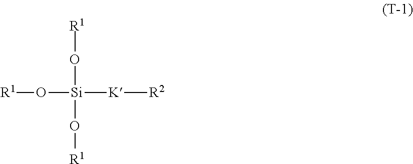
wherein
R¹ is a (C₁ to C₄) alkyl group,
K′ is a connectivity chosen from a covalent bond or from a molecular fragment having two free valences,
R² is a structural fragment having at least one substituent chosen from quaternary ammonium group, tertiary amino group, secondary amino group, primary amino group, and an anionic group.

10. A method for lasting shaping of keratin-containing fibers comprising the steps of:
(i) shaping the fibers with the aid of shaping auxiliary agents after, before or during step (ii),
(ii) bringing an agent according to claim 1 into contact with the fibers for a contact period, and
(iii) optionally rinsing and drying the fibers after a contact period.

11. The method according to claim 10, wherein the contact period is 5 to 20 minutes.

12. The method according to claim 10, further comprising as part of a hair straightening process subjecting the fibers in step (iii) to a heat treatment with mechanical straightening of the fibers at a temperature of 120 to 220° C.
